(19) United States
(12) Patent Application Publication
(10) Pub. No.: US 2026/0295273 A1
(43) Pub. Date: Oct. 1, 2026

(54) BALANCED STIMULATION

(71) Applicant: Cochlear Limited, Macquarie University, NSW (AU)

(72) Inventor: Yves WERNAERS, Affligem (BE)

(21) Appl. No.: 19/477,352

(22) PCT Filed: Apr. 29, 2024
(86) PCT No.: PCT/IB2024/054154

§ 371 Date: Oct. 21, 2025

Related U.S. Application Data
(60) Provisional application No. 63/500,390, filed on May 5, 2023.

(51) Int. Cl. A61N 1/36

(52) U.S. Cl. CPC A61N 1/36157; A61N 1/36175

(57) ABSTRACT

Presented herein are techniques for robust spatially balanced stimulation when, for example, one or more current sources of a medical device run out of compliance. More specifically, a medical device can include one or more current sources configured to deliver electrical stimulation signals (current signals) to a recipient via one or more electrodes. The medical device is configured to determine an out-of-compliance (OOC) condition of at least one of the plurality of current sources during delivery of electrical stimulation signals to the recipient. The medical device is configured to adjust one or more of the electrical stimulation signals in response to the OOC condition.

### BACKGROUND

### Field of the Invention

The present invention relates generally to techniques for balanced stimulation.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: delivering electrical stimulation signals to a recipient via one or more current sources, detecting an out-of-compliance (OOC) condition of a first current source of the one or more current sources during delivery of the electrical stimulation signals, and adjusting one or more of the electrical stimulation signals in response to detecting the OOC condition.

In another aspect, one or more non-transitory computer readable storage media are provided. The one or more non-transitory computer readable storage media comprise instructions that, when executed by one or more processors, cause the one or more processors to: determine an out-of-compliance (OOC) condition of a first current source of an implant that is configured to provide electrical stimulation via one or more electrodes; and stop delivery of at least a first pulse of the electrical stimulation for the one or more electrodes upon detecting the OOC condition.

In another aspect, a method is provided. The method comprises: detecting an out-of-compliance (OOC) condition of a first current source of an implant that is configured to provide electrical stimulation via one or more electrodes; and adapting a width of at least a second pulse of the electrical stimulation for the one or more electrodes after a first pulse during which the OOC condition was detected.

In another aspect, an apparatus is provided. The apparatus comprises: an implant including a stimulator unit configured to deliver electrical stimulation signals to a recipient via one or more current sources; and control circuitry configured to detect an out-of-compliance condition of a first current source of the one or more current sources during delivery of the electrical stimulation signals, and adjust one or more of the electrical stimulation signals in response to detecting the out-of-compliance condition of the first current source.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2A is an example of fully focused multipolar stimulation;

FIG. 2B is an example of louder fully focused multipolar stimulation with increased amplitudes;

FIG. 2C is an example of louder partially focused multipolar stimulation;

FIG. 3A is an example of normal stimulation when the current sources are in compliance;

FIG. 3B is an example of abnormal stimulation behavior when the current source is out-of-compliance (OOC);

FIG. 4 is an example of robust spatially balanced stimulation, even when the current sources are not in compliance, according to an example embodiment;

FIG. 5A is a flowchart of an example method for robust spatially balanced stimulation even when one or more current sources are not in compliance, according to an example embodiment;

FIG. 5B is a flowchart of an example method for robust spatially balanced stimulation even when one or more current sources are not in compliance, according to an example embodiment;

FIG. 5C is a flowchart of an example method for robust spatially balanced stimulation even when one or more current sources are not in compliance, according to an example embodiment; and

FIG. 6 is a schematic diagram illustrating a vestibular stimulator system with which aspects of the techniques presented herein can be implemented.

### DETAILED DESCRIPTION

Presented herein are techniques for robust spatially balanced stimulation when, for example, one or more current sources of a medical device run out of compliance. More specifically, a medical device can include one or more current sources configured to deliver electrical stimulation signals (current signals) to a recipient via one or more electrodes. The medical device is configured to determine an out-of-compliance (OOC) condition of at least one of the plurality of current sources during delivery of electrical stimulation signals to the recipient. The medical device is configured to adjust one or more of the electrical stimulation signals in response to the OOC condition. For example, in response to the OOC condition, the medical device can stop delivery of electrical stimulation signals via at least one (or more) of the one or more electrodes, adapt a pulse width of electrical stimulation signals delivered via at least one (or more) of the one or more electrodes, etc.

There are a number of different types of devices in/with which embodiments of the present invention can be implemented. Merely for ease of description, the techniques presented herein are primarily described with reference to a specific device in the form of a cochlear implant system. However, it is to be appreciated that the techniques presented herein can also be partially or fully implemented by any of a number of different types of devices, including hearing devices, implantable medical devices, consumer electronic devices (e.g., mobile phones), wearable devices (e.g., smart watches), etc. As used herein, the term “hearing device” is to be broadly construed as any device that acts on an actual or potential auditory perception of an individual, including to improve perception of sound signals, to reduce perception of sound signals, etc. In particular, a hearing device can deliver sound signals to a user in any form, including in the form of acoustical stimulation, mechanical stimulation, electrical stimulation, etc., and/or can operate to suppress all or some sound signals. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems, combinations or variations thereof, etc.), a device for use by a person with normal hearing (e.g., consumer devices that provide audio streaming, consumer headphones, earphones, and other listening devices), a hearing protection device, etc. In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

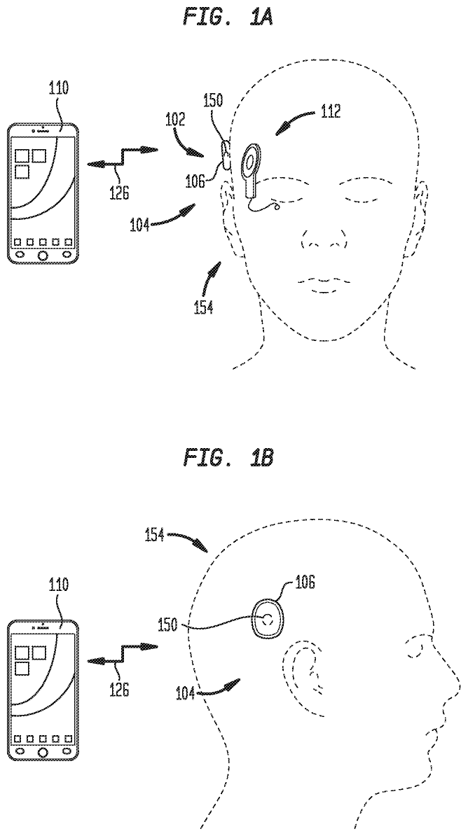

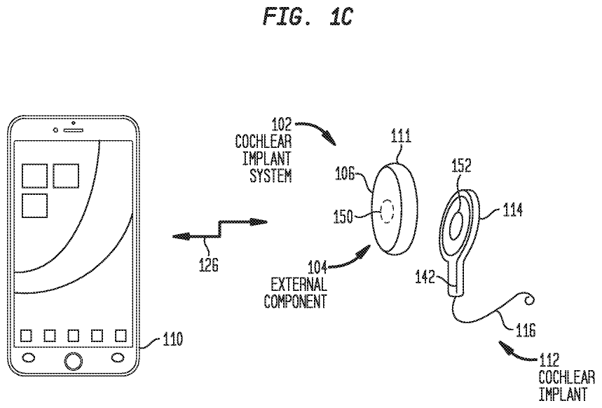

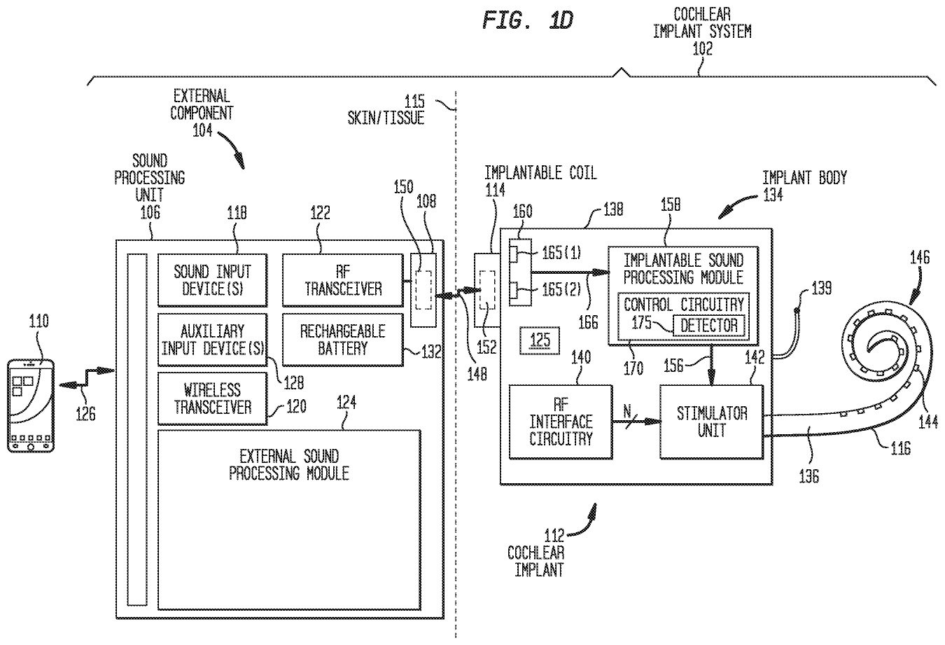

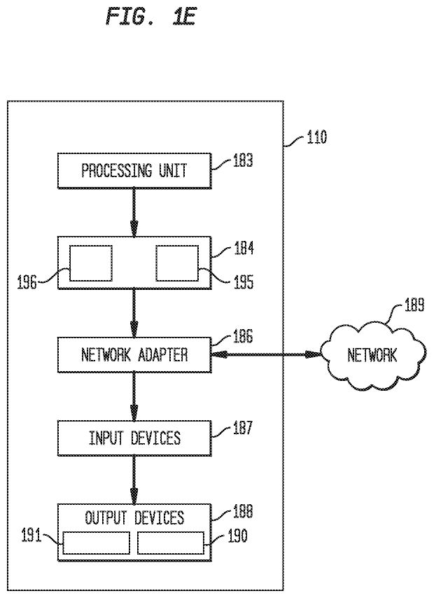

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1D, the implantable component 112 is sometimes referred to as a “cochlear implant.” FIG. 1A illustrates a cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

As detailed below, the cochlear implant system 102 includes an external component. It is to be appreciated that this specific arrangement is merely illustrative and that the techniques presented herein could be implemented by a fully implantable device (e.g., a device that does not include an external component). As such, any reference to external components is for the purpose of explanation and does not limit the techniques presented herein to any specific structural arrangement.

In the examples of FIGS. 1A-1D, the external component 104 comprises a sound processing unit 106, an external coil 108, and generally, a magnet fixed relative to the external coil 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head 154 (e.g., includes an integrated external magnet 150 configured to be magnetically coupled to an internal/implantable magnet 152 in the implantable component 112). The OTE sound processing unit 106 also includes an integrated external (headpiece) coil 108 (the external coil 108) that is configured to be inductively coupled to the implantable coil 114.

It is to be appreciated that the OTE sound processing unit 106 is merely illustrative of the external devices that could operate with implantable component 112. For example, in alternative examples, the external component 104 can comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). Alternatively, the invisible hearing mode may be applied in a variation of an implant system in which there is no external component 104 (no sound processing unit 106 present). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. IE, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices can include additional types of input devices and/or less input devices (e.g., the short-range wireless transceiver 120 and/or one or more auxiliary input devices 128 could be omitted).

The sound processing unit 106 also comprises the external coil 108, a charging coil 130, a closely-coupled radio frequency transmitter/receiver (RF transceiver) 122, at least one rechargeable battery 132, and an external sound processing module 124. The external sound processing module 124 can be configured to perform a number of operations, and can be formed by one or more processors (e.g., one or more Digital Signal Processors (DSPs), one or more uC cores, etc.), firmware, software, etc. arranged to perform operations described herein. That is, the external sound processing module 124 can be implemented as firmware elements, partially or fully implemented with digital logic gates in one or more application-specific integrated circuits (ASICs), partially or fully in software, etc.

Returning to the example of FIGS. 1A-ID, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 that includes, in certain examples, at least one power source 125 (e.g., one or more batteries, one or more capacitors, etc.) 125, in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the internal/implantable magnet 152 is fixed relative to the implantable coil 114. The external magnet 150 and the internal/implantable magnet 152 fixed relative to the external coil 108 and the internal/implantable coil 114, respectively, facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals are provided to the RF transceiver 122, which transcutaneously transfers the output control signals (e.g., in an encoded manner) to the implantable component 112 via external coil 108 and implantable coil 114. That is, the output control signals are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

As also illustrated in FIG. 1D, in certain examples, control circuitry 170, including one or more measurement units/detectors 175, is incorporated into implantable sound processing module 158 of implant body 134. The one or more detectors 175 can each be configured to sense when one or more corresponding current sources have entered into an out-of-compliance (OOC) condition. A current source requires a certain voltage to operate properly, which is referred to as the compliance voltage. Generally, an OOC condition can be detected when the voltage is different from (not the same as) the compliance voltage, and as a result, the current source provides different current than expected. In one non-limiting example, an OOC condition occurs when the voltage drops below the compliance voltage, which causes the current source to provide less current than expected.

Examples of detectors 175 that can be used as part of control circuitry 170 include, for example, one or more current mirrors, capacitors, differential amplifiers, voltage sensors, and the like. Such sensors can be implemented in, for example, micro electromechanical systems (MEMS) or with other technology suitable for the particular application. Although the control circuitry 170 and the detector 175 are shown as part of the implantable sound processing module 158 in FIG. 1D, example embodiments are not limited thereto, and the control circuitry 170 and the detector 175 can alternatively be integrated with the stimulator unit 142, or can be included in a separate and distinct module in the implant body 134 of the cochlear implant 112. For hearing devices that include an implantable sound processing module, such as implantable sound processing module 158, that includes a controller, such as control circuitry 170, the techniques presented herein can be implemented without an external processor. Accordingly, a hearing device that includes an implant body 134 and lacks an external component 104 (no sound processing unit 106 present) can be configured to implement the techniques presented herein.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media or combinations thereof. In certain embodiments, the memory 184 comprises detection logic 195 and stimulation control logic 196 that, when executed, enables the processing unit 183 to perform aspects of the techniques presented. In some example embodiments, the external computing device 110 can be used to program the implantable sound processing module 158 (e.g., the control circuitry 170 and/or the detector 175) with the detection logic 195 and/or the stimulation control logic 196 to enable the implant system to implement the techniques presented herein.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include a display 190 (e.g., a liquid crystal display (LCD)) and one or more speakers 191, among other output devices for presentation of visual or audible information to the recipient, a clinician, an audiologist, or other user.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

As noted, presented herein are techniques for robust spatially balanced stimulation even when, for example, one or more current sources run out of compliance. Spatially balanced stimulation, also referred to as multipolar stimulation, can be used for current steering or current focusing and the sum of all current must be zero. Stated differently, the sum of all delivered current should be zero under normal operating conditions when one or more current sources are operating properly.

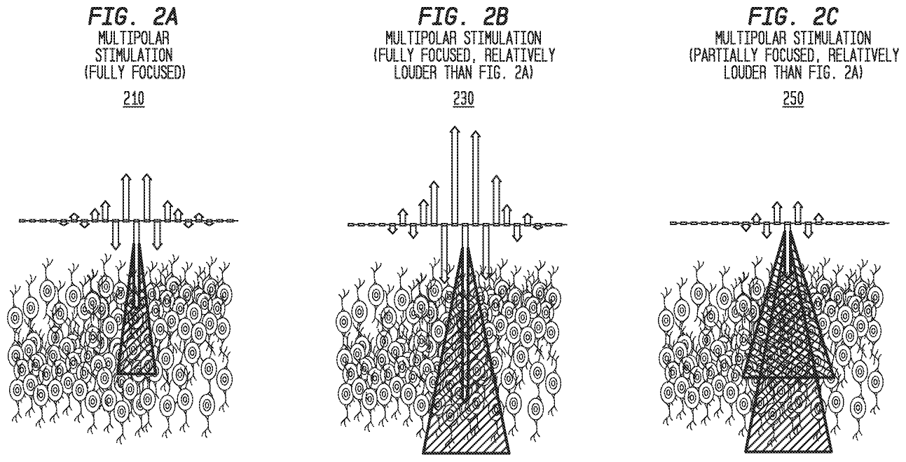

FIG. 2A is a diagram illustrating an example of fully focused multipolar stimulation 210, while FIG. 2B is an example of relatively louder fully focused multipolar stimulation 230, with increased amplitudes. FIG. 2C is an example of relatively louder partially focused multipolar stimulation 250. The arrows in FIGS. 2A-2C represent the amplitudes of the electrical stimulation signals/current (where the sum equals, or should equal, zero).

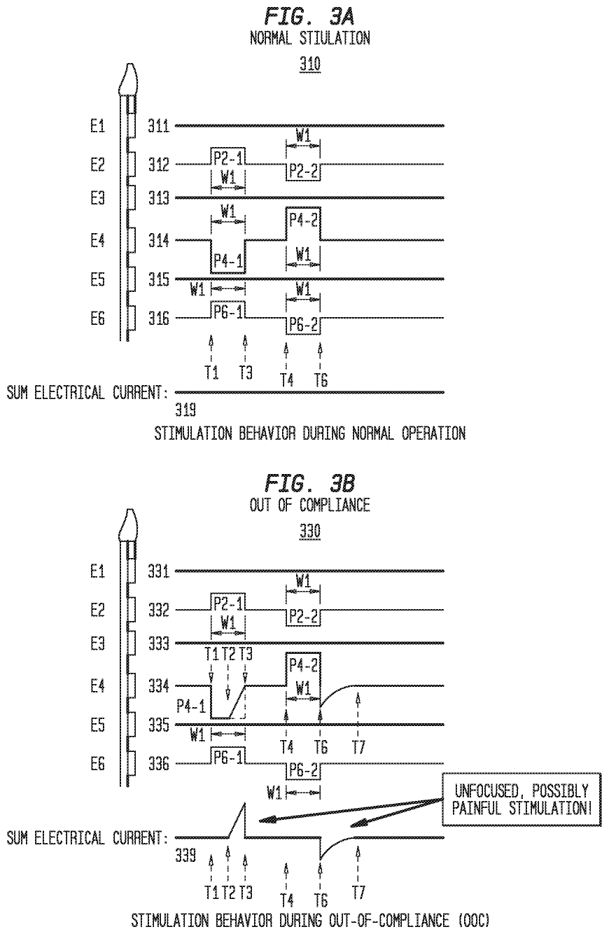

FIG. 3A is an example of normal stimulation 310 when the current sources are in compliance. Merely for ease of illustration, FIG. 3A shows six electrodes E1, E2, E3, E4, E5, and E6 and six stimulation signal waveforms 311, 312, 313, 314, 315, and 316, respectively. There are three driving currents in this example, represented by waveform 312 corresponding to electrode E2, waveform 314 corresponding to electrode E4, and waveform 316 corresponding to electrode E6. The sum of all current (stimulation signals) should be zero at any given time during the stimulation.

In the example of FIG. 3A, the amplitude of the first pulse P4-1 should equal the sum of the amplitudes of the first pulse P2-1 and the first pulse P6-1, and the amplitude of the second pulse P4-2 should equal the sum of the amplitudes of the second pulse P2-2 and the second pulse P6-2, (and therefore these pulses should cancel each other out). The first pulses P2-1, P4-1, and P6-1 have a pulse width W1 (a time duration from time T1 to time T3), and the second pulses P2-2, P4-2, and P6-2 have a pulse width W1 (a time duration from time T4 to time T6). The sum electrical current 319 as shown in FIG. 3A is zero. Thus, FIG. 3A is an example in which the expected current (set current) from the current source is the same as the actual current being delivered (measured current, or calculated current).

As noted, a current source requires a certain voltage to operate properly, referred to as the compliance voltage. When the voltage drops below the compliance voltage, the current source provides less current, which is referred to herein as an “out-of-compliance” (OOC) condition.

FIG. 3B is an example of abnormal stimulation behavior 330 when a current source is out-of-compliance (OOC). FIG. 3B shows six electrodes E1, E2, E3, E4, E5, and E6 and six stimulation signal waveforms 331, 332, 333, 334, 335, and 336, respectively. There are three driving currents in this example, represented by waveform 332 corresponding to electrode E2, waveform 334 corresponding to electrode E4, and waveform 336 corresponding to electrode E6. The sum of all current should be zero at any given time during the stimulation, but is not zero at all times in this example.

In the example of FIG. 3B, the amplitude of the first pulse P4-1 does not equal the sum of the amplitudes of the first pulse P2-1 and the first pulse P6-1 (and therefore these pulses do not cancel each other out). The first pulses P2-1 and P6-1 have a pulse width W1 (a time duration from time T1 to time T3), and the second pulses P2-2 and P6-2 have a pulse width W1 (a time duration from time T4 to time T6); however, the first pulse P4-1 corresponding to the electrode E4 does not have the corresponding pulse width. With respect to the first pulse, the current source associated with electrode E4 enters an out-of-compliance (OOC) condition at time T2 during the first pulse. The time duration from time T6 to time T7 corresponds to a charge neutralization phase, which is an integral part of charge balanced stimulation. The total integrated current (=charge) of the second pulse P4-2 is not the same as the integrated current of the first pulse P4-1, which is why at the end of the second pulse, there is a current flowing from electrode E4 during the charge neutralization phase (from T6 to T7). The sum electrical current 339 in FIG. 3B is non-zero for two periods of time (e.g., from time T2 to time T3 during the first pulse, and from time T6 to time T7 after the second pulse). The spikes in the sum electrical current 339 can result in, or represent, unfocused and possibly painful stimulation. Thus, FIG. 3B is an example in which the expected current (set current) from the current source is not the same as (e.g., is less than) the actual current being delivered (measured current, or calculated current).

Over time during the first pulse, voltage builds up because current is being sent, and part of the cochlea is capacitive (i.e., the more current being driven, the larger the voltage across the capacitance becomes). So over time, a certain voltage can be reached where the current source cannot actually drive the current needed anymore (the intended stimulation current is not actually being delivered). Getting out of compliance is represented by the sloped line of waveform 334 from time T2 to time T3 in FIG. 3B. There is no longer a flat current, as represented by the sum electrical current 339 in FIG. 3B. The driving current reduces towards zero over time, as represented by the end of the diagonal line at time T3 in FIG. 3B. The OOC condition typically occurs on the one or more electrode(s) that are driving most of the current (largest amplitude, highest current level).

During normal stimulation, the currents are balanced such that stimulation is focused to a specific region in the cochlea. However, when a current source is out-of-compliance, this focusing is invalid, and suddenly there are currents going everywhere (and not really where intended), because the implant system is not actually driving the current that is expected. At the end of the second pulse, the remainder of the current (residual current that is not driven by all the DACS) is going/flowing anywhere (e.g., wherever it finds the lowest ohmic part, the current goes there). This is a controlled method to perform charge neutralization, whereby all of the electrodes are being connected to redistribute the residual charges built up in the capacitances. Charges going anywhere are redistributed to all electrodes, which is represented by the curved line of waveform 334 from time T6 to time T7 in FIG. 3B. For example, there can be an RC discharge effect on the electrode E4 (i.e., the RC-discharge on E4 is somehow forced to flow to all of the other electrodes). Again, there is no longer a flat current, as represented by the sum electrical current 339 in FIG. 3B. The driving current reduces towards zero over time, as represented by the end of the curved line at time T7 in FIG. 3B. Since the currents are unbalanced (imbalanced, unbalanced), this could potentially cause a lot of pain to the implant recipient, depending on where the current flows (especially for multipolar stimulation, which can have about 4× higher stimulation currents than monopolar stimulation, for example).

In other words, when a current source is not working properly, stimulation compliance issues can cause spatially imbalanced stimulation, which can result in painful stimulation (especially when using multipolar stimulation, in particular). Because multipolar stimulation has relatively high stimulation currents, and much higher stimulation currents than monopolar stimulation (e.g., about 4 times higher), the OOC condition can occur more frequently (i.e., multipolar implants can be more susceptible to OOC current sources). There are various reasons why a current source can go out of compliance, such as varying tissue and cochlear impedance, or slow adaptive stimulation voltage regulation (e.g., stimulation voltage is not increased fast enough when the implant recipient goes from a quiet environment to a loud environment, suddenly causing much higher stimulation amplitudes).

According to one current technique for avoiding unfocused and/or painful stimulation in the case of using multipolar stimulation, the entire implant system resets if the current source goes out-of-compliance. In this instance, referring to the example of FIG. 3B, the implant system only sends part of the first phase (a partial first pulse), and does not send the second phase at all (no second pulse). In future systems where power must be kept as low as possible, such as multipolar implants that provide multipolar stimulation, OOC will occur more frequently when recipients go from a quiet environment to a loud environment (e.g., as the power supply will be kept at the edge of compliance), which currently would cause an implant reset (hence the recipient remains without sound for a few seconds).

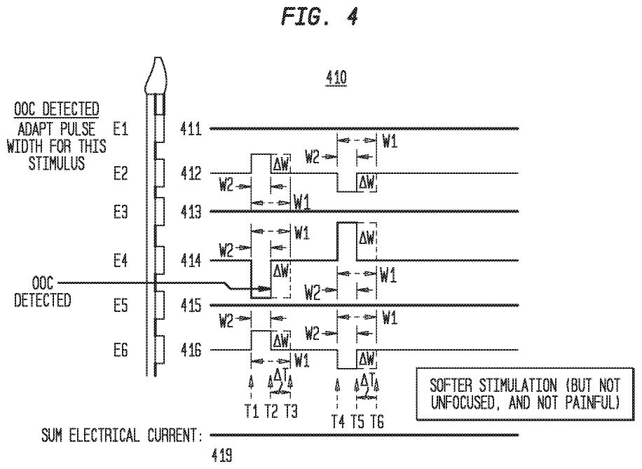

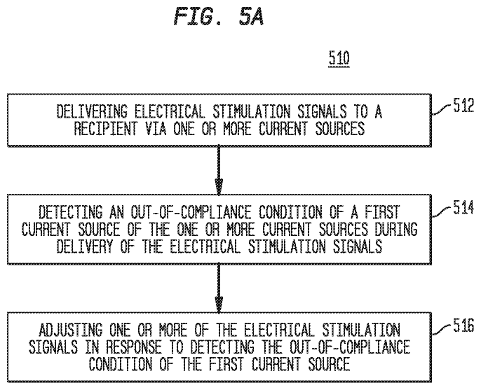

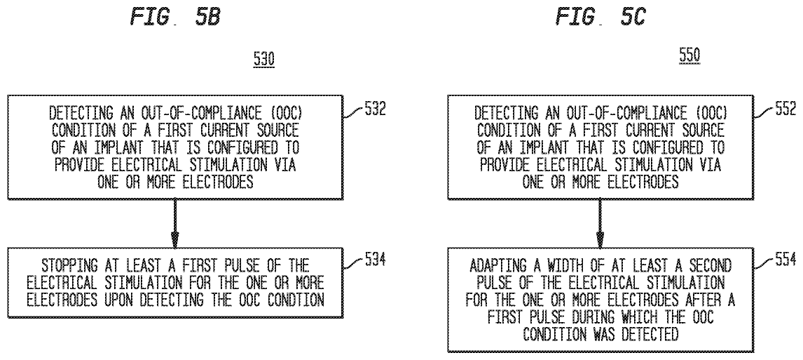

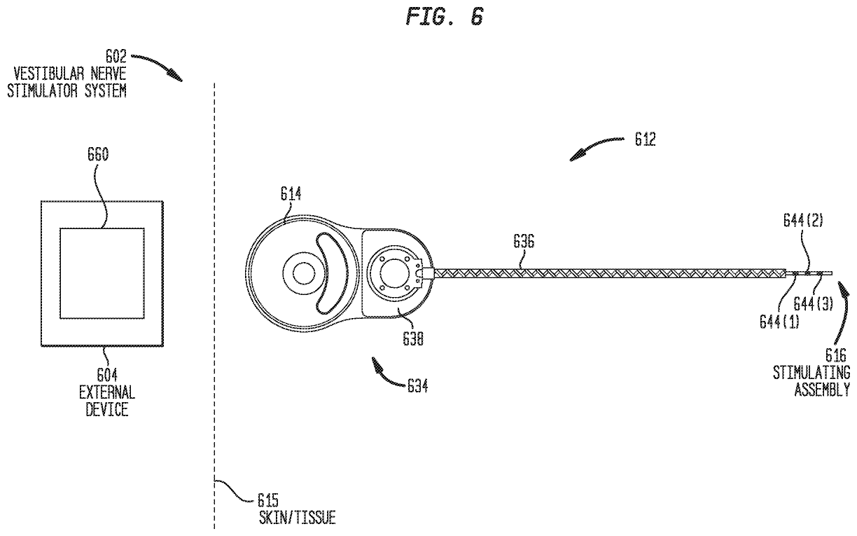

According to an aspect of the present disclosure, a system and methods for robust spatially balanced stimulation are provided, as described below with reference to FIGS. 4 and 5A-5C, respectively. The implant system and methods can leverage the detector 175 (“OOC detector”) to identify when a current source goes into the out-of-compliance condition (e.g., determine whether current deviation between the set/expected current level and the actual/measured/calculated current level is greater than a 1-2% margin of error, for example). As mentioned above, non-limiting examples of the detector 175 can include one or more current mirrors, measurement capacitors, differential amplifiers, or other components and circuitry. The control circuitry 170 can then control the stimulator unit 142 according to the techniques presented herein.

FIG. 4 is an example of robust spatially balanced stimulation 410, even when one or more of the current sources are not in compliance, according to an example embodiment. FIG. 4 shows six electrodes E1, E2, E3, E4, E5, and E6 and six stimulation signal waveforms 411, 412, 413, 414, 415, and 416, respectively. There are three driving currents in this example, represented by waveform 412 corresponding to electrode E2, waveform 414 corresponding to electrode E4, and waveform 416 corresponding to electrode E6. The sum of all current should be zero at any given time during the stimulation.

In the example of FIG. 4, the amplitude of the first pulse in waveform 414 for electrode E4 should equal the sum of the amplitudes of the first pulse in waveform 412 and the first pulse in waveform 416, and the amplitude of the second pulse in waveform 414 should equal the sum of the amplitudes of the second pulse in waveform 412 and the second pulse in waveform 416, (and therefore these pulses should cancel each other out). The first pulses have a set pulse width W1 (a time duration from time T1 to time T3) that is reduced to a pulse width W2 (a time duration from time T1 to time T2), and the second pulses also have the set pulse width W1 (a time duration from time T4 to time T6) that is also reduced to the pulse width W2 (a time duration from time T4 to time T5). The sum electrical current 419 as shown in FIG. 4 remains (is maintained) at zero according to the techniques described herein. Thus, FIG. 4 is an example in which the actual current being delivered (measured current, or calculated current) is less/lower than the expected current (set current) from the current source, but the stimulation remains charge-balanced at any given time throughout the stimulation.

In a first phase (i.e., for first pulses corresponding to a first electrode and one or more other electrodes), in response to detecting that a current source of the implant system is out-of-compliance (OOC), as represented in waveform 414 at time T2, the system will prematurely stop driving current for that particular pulse (e.g., send approximately half a pulse) such that the first pulse has a reduced pulse width W2 (a shorter time duration from time T1 to time T2), but does not stop or reset the entire implant system (i.e., just stopping the OOC part of the stimulation—a first electrode plus one or more other electrodes). The system stops the first pulse immediately at that moment in time (e.g., time T2) when the OOC condition is detected. In FIG. 4, the difference in pulse width between the set pulse width W1 and the actual pulse width W2 is represented by ΔW, and the difference in time duration between the set pulse and the actual pulse is represented by ΔT. Any of the reduced pulse width W2, the time T2, the difference in pulse width ΔW, or the difference in time duration ΔT can be measured or calculated for further use in the second phase (for the second pulse).

Then the system does the same thing for the second phase (i.e., for second pulses corresponding to the first electrode and the other electrodes). The second pulse (i.e., the next/subsequent pulse after the first pulse that was stopped upon detecting the OOC condition) is adapted/adjusted based on the stopped first pulse (i.e., based on the reduced pulse width W2, based on the reduced time duration from T1 to T2, based on the pulse width difference ΔW between W1 and W2, and/or based on the time difference ΔT between T2 and T3).

The time duration for the first pulse can be measured or calculated (e.g., an actual duration of the first pulse for waveform 414 that was stopped short from time T1 to time T2, or a difference ΔT between the expected stop time T3 and the actual stop time T2 for the first pulse of waveform 414). This measured/calculated time, also referred to herein as a pulse width, can then be used to adjust the pulse width (or time duration) of the second pulse to match that of the first pulse. Stopping the first pulse prematurely, and adjusting the second pulse accordingly based thereon, can result in ‘softer’ stimulation (due to the shorter pulse width/time duration), but with the advantage that the stimulation is not unfocused and is not painful.

In one non-limiting illustrative example, the system can set pulse time duration (pulse width) at 50 microseconds, but detects an OOC condition and immediately stop a first pulse after only 20 microseconds. The system can know that the first pulse was stopped after 20 microseconds (actual time, actual pulse width), instead of 50 microseconds (expected time, expected pulse width). The system can then control the second pulse after detecting the OOC condition to have a pulse time (pulse width) of 20 microseconds (instead of 50 microseconds) in order to match the first pulse that was prematurely stopped by the system. Of course, numerous varying timings and pulse widths can be used in practice to adapt the techniques described here to real-world scenarios.

These techniques effectively compensate for the OOC condition (at least temporarily), and enable the implant system to maintain balanced, focused stimulation. Although stopping and/or adapting/adjusting the time duration (pulse width) results in softer sound for the first and second pulses, this technique does not result in defocusing (unfocused stimulation) and also avoids painful stimulation (unbalanced stimulation) despite the presence of an OOC current source.

It should be noted that the amount or degree to which the time durations (pulse widths) of the first and second pulses are reduced can depend on, and can vary based on, an amount of current being driven (e.g., louder vs softer sounds) in addition to various other physiological factors (e.g., impedances of implant system). A half pulse (50% pulse width) is just one non-limiting illustrative example, and it could be a quarter pulse (25% pulse width), or a three-quarter pulse (75% pulse width), or any other ratio (e.g., from 1-99% pulse width). If a lower current level is being driven, then the stopped and the adapted/adjusted times (pulse widths) can be larger (i.e., closer to the expected setting). If a higher current level is being driven, then the stopped and the adapted/adjusted times (pulse widths) can be smaller (i.e., farther from the expected setting).

In an example embodiment, the OOC condition of the current source can also be corrected for or eliminated in a further operation, by increasing the voltage to the DAC for later phases/pulses of the stimulation. This can be performed for a third pulse, fourth pulse, etc. of the stimulation (i.e., any other later pulse after the first pulse that is stopped prematurely, and the second pulse that is adapted/adjusted/reduced accordingly).

Although some example embodiments shown in the figures and described above show a first phase (first pulse) and a second phase (second phase) being relatively the same or similar in amplitude (current level) and/or pulse width (time duration), this is merely for simplicity and ease of explanation and it should be appreciated that the techniques described herein can be similarly applied in use cases where the first phase/pulse and the second phase/pulse have differing amplitudes (higher or lower current levels, respectively), differing pulse widths (shorter or longer time durations, respectively), or both. Although a certain number of electrodes or waveforms or current sources, etc. can be described, example embodiments are not limited thereto and there can be fewer or greater components involved. Additionally or alternatively, some other example embodiments can involve creating a lesser amplitude (lower current level) and/or a larger pulse width (longer duration) for the second phase of the stimulation to avoid unbalanced stimulation.

In another example embodiment, the implant system can be configured to adjust the stimulation voltage over or after a longer period of time, which can allow the implant system to adapt stimulation as appropriate, such as due to changes in the impedance of the cochlea of the recipient over time as the recipient ages.

Another advantage of the system and techniques described herein is to keep the voltage of the implant as low as possible (minimize power consumption, optimize the stimulation voltage). Power efficiencies can be gained by not always generating the highest stimulation voltage possible or allowed. The system can utilize a first lower voltage level while the implant recipient is in a quiet environment. When the implant recipient transitions into a loud environment, the system can detect an OOC condition, prematurely stop the first pulse immediately upon detecting the OOC condition, and adapt/adjust (i.e., reduce) the second pulse after detecting the OOC condition. The implant recipient will then hear softer sounds for a short period of time (e.g., the first and second pulses). In addition, the system can then increase the voltage to a second higher level for later pulses (e.g., third, fourth, etc.) to correct for or eliminate the OOC condition while the recipient is in the loud environment. Likewise, the system can detect when the implant recipient leaves the loud environment and returns to a quiet environment, at which point the system can then reduce the voltage back down to the first lower voltage level. In addition, there could be various intermediate voltage levels so that the voltage can vary with increasing and decreasing sound levels in the surrounding environment. Thus, the system can dynamically adjust the voltage to adapt to the sound level (loudness) in the surrounding environment of the implant recipient. There is no reset operation of the implant system, and there is no large power consumption which would otherwise Cooccur if the system is driving the higher voltage level.

Next, several example methods for robust spatially balanced stimulation, even when one or more current sources are not in compliance, using the system and techniques presented herein are described with reference to FIGS. 5A-5C.

FIG. 5A is a flowchart of an example method 510 for robust spatially balanced stimulation even when one or more current sources are not in compliance, according to an example embodiment. At operation 512, method 510 includes delivering electrical stimulation signals to a recipient via one or more current sources. At operation 514, method 510 includes detecting an out-of-compliance condition of a first current source of the one or more current sources during delivery of the electrical stimulation signals. At operation 516, method 510 includes adjusting one or more of the electrical stimulation signals in response to detecting the out-of-compliance condition of the first current source.

In some examples, adjusting the one or more electrical stimulation signals in response to detecting the out-of-compliance condition of the first current source includes adjusting a pulse width of the one or more electrical stimulation signals. In some examples, delivering the electrical stimulation signals includes delivering a first electrical stimulation signal from the first current source via a first electrode, and adjusting the pulse width of the one or more electrical stimulation signals includes stopping the first electrical stimulation signal upon detecting the out-of-compliance condition. In some examples, the first electrical stimulation signal, when stopped, has a shortened pulse width, and adjusting the pulse width of the one or more electrical stimulation signals further includes delivering a subsequent pulse from the first current source via the first electrode with the shortened pulse width, wherein the subsequent pulse has an opposite polarity to the first pulse. In some examples, the electrical stimulation signals comprise multipolar electrical stimulation signals that further include a second electrical stimulation signal delivered contemporaneously with the first electrical stimulation signal from a second current source via a second electrode, and adjusting the pulse width of the one or more electrical stimulation signals further includes stopping the second electrical stimulation signal upon detecting the out-of-compliance condition. In some examples, the second electrical stimulation signal, when stopped, has a shortened pulse width, and adjusting the pulse width of the one or more electrical stimulation signals further includes delivering a subsequent pulse from the second current source via the second electrode with the shortened pulse width, wherein the subsequent pulse has an opposite polarity to the second pulse.

FIG. 5B is a flowchart of an example method 530 for robust spatially balanced stimulation even when one or more current sources are not in compliance, according to an example embodiment. At operation 532, method 530 includes detecting an out-of-compliance (OOC) condition of a first current source of an implant that is configured to provide electrical stimulation via one or more electrodes. At operation 534, method 530 includes stopping at least a first pulse of the electrical stimulation for the one or more electrodes upon detecting the OOC condition.

In some examples, detecting the OOC condition includes measuring the current generated by the first current source, and determining that the current delivered by the first current source is lower than a predetermined amount. In some examples, the implant continues to operate upon detecting the OOC condition. In some examples, stopping the first pulse of the stimulus includes immediately cutting off the first pulse at a moment in time at which the OOC condition is detected for the first current source. In some examples, stopping the first pulse of the electrical stimulation for the one or more electrodes includes stopping a first pulse for a first electrode of an electrode array, for which the OOC condition of the first current source is detected, and stopping one or more first pulses for one or more other electrodes of the electrode array. In some examples, the method can further include adapting a width of at least a second pulse of the electrical stimulation for the one or more electrodes after detecting the OOC condition of the first current source. In some examples, adapting the width of the second pulse of the electrical stimulation for the one or more electrodes further includes adapting a width of a second pulse for a first electrode of an electrode array, for which the OOC condition of the first current source is detected, and adapting one or more widths of one or more second pulses for one or more other electrodes of the electrode array. In some examples, adapting the width of the second pulse comprises reducing the width of the second pulse based on a width of the first pulse.

FIG. 5C is a flowchart of an example method 550 for robust spatially balanced stimulation even when one or more current sources are not in compliance, according to an example embodiment. At operation 552, method 550 includes detecting an out-of-compliance (OOC) condition of a first current source of an implant that is configured to provide electrical stimulation via one or more electrodes. At operation 554, method 550 includes adapting a width of at least a second pulse of the electrical stimulation for the one or more electrodes after a first pulse during which the OOC condition was detected.

In some examples, detecting the OOC condition comprises measuring the current generated by the first current source, and determining that the current delivered by the first current source is lower than a predetermined amount. In some examples, the implant continues to operate upon detecting the OOC condition. In some examples, the method can further include stopping at least the first pulse of the electrical stimulation for the one or more electrodes upon detecting the OOC condition. In some examples, stopping the first pulse of the stimulus includes immediately cutting off the first pulse at a moment in time at which the OOC condition is detected for the first current source. In some examples, stopping the first pulse of the electrical stimulation for the one or more electrodes includes stopping a first pulse for a first electrode of an electrode array, for which the OOC condition of the first current source is detected, and stopping one or more first pulses for one or more other electrodes of the electrode array. In some examples, adapting the width of the second pulse of the electrical stimulation for the one or more electrodes includes adapting a width of a second pulse for a first electrode of an electrode array, for which the OOC condition of the first current source is detected, and adapting one or more widths of one or more second pulses for one or more other electrodes of the electrode array. In some examples, adapting the width of the second pulse comprises reducing the width of the second pulse based on a width of the first pulse.

It is noted that the example methods 510, 530, and 550 of FIGS. 5A, 5B, and 5C can cause a ‘softer’ stimulus, but there is no spatial imbalance, and hence, there is no painful stimulation. Stated differently, the implant remains stimulating spatially balanced (or charge-balanced, even though there is an OOC current source), hence there is no painful stimulation, nor does the implant need to reset due to the out-of-compliance condition.

Applying the mechanisms described above allows for advanced power reduction schemes without having the penalty of resetting the implant or causing painful stimulation to the recipient. The techniques described herein can aid in avoiding unbalanced stimulation while keeping the supply voltage of the implant as low as possible. This allows for more efficient operation of an implant, and is particularly useful for multipolar implants (multipolar stimulation), for which power consumption is a significant concern.

As previously described, the technology disclosed herein can be applied in any of a variety of circumstances and with a variety of different devices. Example devices that can benefit from technology disclosed herein are described in more detail in FIG. 6. The techniques of the present disclosure can be applied to other devices, such as neurostimulators (e.g., fully implantable neurostimulators), cardiac pacemakers, cardiac defibrillators, sleep apnea management stimulators, seizure therapy stimulators, tinnitus management stimulators, and vestibular stimulation devices, as well as other medical devices that deliver stimulation to tissue. Furthermore, the technology described herein can also be applied to consumer devices. These different systems and devices can benefit from the technology described herein.

FIG. 6 illustrates an example vestibular stimulator system 602, with which embodiments presented herein can be implemented. As shown, the vestibular stimulator system 602 comprises an implantable component (vestibular stimulator) 612 and an external device 604 (e.g., external processing device, battery charger, remote control, component, etc.). The external device 604 comprises a transceiver unit 660. As such, the external device 604 is configured to transfer data (and potentially power) to the vestibular stimulator 612.

The vestibular stimulator 612 comprises an implant body (main module) 634, a lead region 636, and a stimulating assembly 616, all configured to be implanted under the skin/tissue (tissue) 615 of the recipient. The implant body 634 generally comprises a hermetically-sealed housing 638 in which RF interface circuitry, one or more rechargeable batteries, one or more processors, and a stimulator unit are disposed. The implant body 634 also includes an internal/implantable coil 614 that is generally external to the housing 638, but which is connected to the transceiver via a hermetic feedthrough (not shown).

The stimulating assembly 616 comprises a plurality of electrodes 644(1)-(3) disposed in a carrier member (e.g., a flexible silicone body). In this specific example, the stimulating assembly 616 comprises three (3) stimulation electrodes, referred to as stimulation electrodes 644(1), 644(2), and 644(3). The stimulation electrodes 644(1), 644(2), and 644(3) function as an electrical interface for delivery of electrical stimulation signals to the recipient's vestibular system.

The stimulating assembly 616 is configured such that a surgeon can implant the stimulating assembly adjacent to the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 612, the external device 604, and/or another external device, can be configured to implement the techniques presented herein. That is, the vestibular stimulator 612, possibly in combination with the external device 604 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure. In some examples, however, the techniques described herein may be implemented completely in hardware of an internal/implantable component (without requiring an external component or external computing device with instructions and a processor).

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
delivering electrical stimulation signals to a recipient via one or more current sources;
detecting an out-of-compliance condition of a first current source of the one or more current sources during delivery of the electrical stimulation signals; and
adjusting one or more of the electrical stimulation signals in response to detecting the out-of-compliance condition of the first current source.

2. The method of claim 1, adjusting the one or more electrical stimulation signals in response to detecting the out-of-compliance condition of the first current source comprises:
adjusting a pulse width of the one or more electrical stimulation signals.

3. The method of claim 2, wherein delivering the electrical stimulation signals includes delivering a first electrical stimulation signal from the first current source via a first electrode, and wherein adjusting the pulse width of the one or more electrical stimulation signals comprises:
stopping delivery of the first electrical stimulation signal upon detecting the out-of-compliance condition.

4. The method of claim 3, wherein the first electrical stimulation signal, when delivery is stopped, has a shortened pulse width, and wherein adjusting the pulse width of the one or more electrical stimulation signals further comprises:
delivering a subsequent pulse from the first current source via the first electrode with the shortened pulse width,
wherein the subsequent pulse has an opposite polarity to the first pulse.

5. The method of claim 3, wherein the electrical stimulation signals comprise multipolar electrical stimulation signals that further include a second electrical stimulation signal delivered contemporaneously with the first electrical stimulation signal from a second current source via a second electrode, and wherein adjusting the pulse width of the one or more electrical stimulation signals further comprises:
stopping delivery of the second electrical stimulation signal upon detecting the out-of-compliance condition.

6. The method of claim 5, wherein the second electrical stimulation signal, when delivery is stopped, has a shortened pulse width, and wherein adjusting the pulse width of the one or more electrical stimulation signals further comprises:
delivering a subsequent pulse from the second current source via the second electrode with the shortened pulse width,
wherein the subsequent pulse has an opposite polarity to the second pulse.

7. One or more non-transitory computer readable storage media comprising instructions that, when executed by one or more processors, cause the one or more processors to:
determine an out-of-compliance (OOC) condition of a first current source of an implant that is configured to provide electrical stimulation via one or more electrodes; and
stop delivery of at least a first pulse of the electrical stimulation for the one or more electrodes upon detecting the OOC condition.

8. The one or more non-transitory computer readable storage media of claim 7, wherein the instructions to determine the OOC condition comprise instructions that, when executed by, the one or more processors, cause the one or more processors to:
obtain a measurement of current generated by the first current source; and
determine that the current delivered by the first current source is lower than a predetermined amount.

9. The one or more non-transitory computer readable storage media of claim 7, wherein the implant continues to operate upon detecting the OOC condition.

10. The one or more non-transitory computer readable storage media of claim 7, wherein the instructions to stop delivery of the first pulse of the electrical stimulation comprises instructions that, when executed by, the one or more processors, cause the one or more processors to:
cut off the first pulse at a moment in time at which the OOC condition is detected for the first current source.

11. The one or more non-transitory computer readable storage media of claim 7, wherein the instructions to stop delivery of the first pulse of the electrical stimulation comprises instructions that, when executed by, the one or more processors, cause the one or more processors to:
stop delivery of a first pulse for a first electrode of an electrode array, for which the OOC condition of the first current source is detected; and
stop delivery of one or more first pulses for one or more other electrodes of the electrode array.

12. The one or more non-transitory computer readable storage media of claim 7, further comprising instructions that, when executed by the one or more processors, cause the one or more processors to:
adapt a width of at least a second pulse of the electrical stimulation for the one or more electrodes after detecting the OOC condition of the first current source.

13. The one or more non-transitory computer readable storage media of claim 12, wherein the instructions to adapt the width of the second pulse of the electrical stimulation for the one or more electrodes further comprises instructions that, when executed by, the one or more processors, cause the one or more processors to:
adapt a width of a second pulse for a first electrode of an electrode array, for which the OOC condition of the first current source is detected; and
adapt one or more widths of one or more second pulses for one or more other electrodes of the electrode array.

14. The one or more non-transitory computer readable storage media of claim 12, wherein the instructions to adapt the width of the second pulse comprise instructions that, that, when executed by, the one or more processors, cause the one or more processors to:
reduce the width of the second pulse based on a width of the first pulse.

15-22. (canceled)

23. An apparatus comprising:
an implant including a stimulator unit configured to deliver electrical stimulation signals to a recipient via one or more current sources; and
control circuitry configured to:
detect an out-of-compliance condition of a first current source of the one or more current sources during delivery of the electrical stimulation signals; and
adjust one or more of the electrical stimulation signals in response to detecting the out-of-compliance condition of the first current source.

24. The apparatus of claim 23, the control circuitry is configured to:
adjust a pulse width of the one or more electrical stimulation signals.

25. The apparatus of claim 24, wherein delivering the electrical stimulation signals includes delivering a first electrical stimulation signal from the first current source via a first electrode, and wherein to adjust the pulse width of the one or more electrical stimulation signals, the control circuitry is configured to control the stimulator unit to stop the first electrical stimulation signal upon detecting the out-of-compliance condition.

26. The apparatus of claim 25, wherein the first electrical stimulation signal, when stopped, has a shortened pulse width, and wherein to adjust the pulse width of the one or more electrical stimulation signals, the control circuitry is further configured to control the stimulator unit to deliver a subsequent pulse from the first current source via the first electrode with the shortened pulse width, wherein the subsequent pulse has an opposite polarity to the first pulse.

27. The apparatus of claim 25, wherein the electrical stimulation signals comprise multipolar electrical stimulation signals that further include a second electrical stimulation signal delivered contemporaneously with the first electrical stimulation signal from a second current source via a second electrode, and wherein to adjust the pulse width of the one or more electrical stimulation signals, the control circuitry is further configured to control the stimulator unit to stop the second electrical stimulation signal upon detecting the out-of-compliance condition.

28. The apparatus of claim 27, wherein the second electrical stimulation signal, when stopped, has a shortened pulse width, and wherein to adjust the pulse width of the one or more electrical stimulation signals, the control circuitry is further configured to control the stimulator unit to deliver a subsequent pulse from the second current source via the second electrode with the shortened pulse width, wherein the subsequent pulse has an opposite polarity to the second pulse.
